(19) United States
(12) Patent Application Publication
(10) Pub. No.: US 2025/0032312 A1
(43) Pub. Date: Jan. 30, 2025

(54) OCULAR DRUG TRANSFER AND DISPENSING DEVICE

(71) Applicant: University of Miami, Coral Gables, FL (US)

(72) Inventor: Alfonso L. SABATER, Miami, FL (US)

(21) Appl. No.: 18/713,960

(22) PCT Filed: Nov. 28, 2022
(86) PCT No.: PCT/IB2022/061499

§ 371 Date: May 28, 2024

Related U.S. Application Data
(60) Provisional application No. 63/283,944, filed on Nov. 29, 2021.

(51) Int. Cl. A61F 9/00; A61J 1/20

(52) U.S. Cl. CPC A61F 9/0008; A61J 1/2048; A61J 1/2096

(57) ABSTRACT

Devices, systems, and methods for transferring, storing, and/or dispensing A fluid, such as an ocular drug. In one embodiment, a device includes: a first end; and a Second end opposite the first end, the first end being configured to be engageable with a Secondary reservoir and the second end being configured to release a fluid therefrom. In one Embodiment, a method of administering a product includes engaging a secondary reservoir With an engagement assembly at a first end of a device; and delivering a fluid from the Secondary reservoir through the engagement assembly and into a chamber of the device to Mix with a product within the chamber.

### technical field

this disclosure relates to a fluid transfer and dispensing device, such as an ocular drug Transfer and dispensing device. In one non-limiting example, a fluid transfer and dispensing Device may be used for preparing and/or administering a product, such as a reconstituted Lyophilized (freeze-dried) or powdered material.

### background

certain ocular disorders, including dry eye, corneal ulcers, and persistent epithelial defects, May be treated with products and medications delivered as eye drops. Many of these biological Products, such as those derived from serum or plasma, have a limited shelf life. However, Many such ocular disorders require treatment over a long period of time, and longevity of the Treatment product is highly desired. Additionally, products that are not cold-chain dependent Are advantageous. Freeze drying, or lyophilization, has been found to be a useful process for Producing treatment products that do not require refrigeration and that have an extended shelf Life.

to administer a lyophilized material (which may be powdered in form) as an eye drop, the Lyophilized material must first be reconstituted with a fluid. There are currently two commonly Used ways of achieving reconstitution: (1) mixing the powdered material with a diluent in a Single-chamber vial; and (2) mixing the powdered material and diluent in a double-chamber Syringe. In the former method, the material and diluent must either be mixed in advance of use And then packaged in a sterile container (which may now require refrigeration of the Reconstituted product and/or reduce its shelf life) or the user must mix the powdered material And diluent at the time of use (which may introduce contaminants and user error). In the latter Method, the powdered material and diluent are housed in separate chambers and then mixed When the plunger of the syringe is depressed. Although this allows for mixture in a sterile Environment, there is currently no good way to transfer the reconstituted product into an eye Dropper for administration. For example, to fill an eye dropper, one would remove a cap/teat From the eye dropper and then pour/add the reconstituted product. However, that method risks Contamination.

additionally, preserving sterility is a concern when preparing and/or administering many Medical products, regardless of whether the products are lyophilized and/or require Reconstitution. For example, the processes used by compounding pharmacies may require steps That risk the introduction of contaminants into open containers and/or into dispensing devices.

### summary

some embodiments advantageously provide devices, systems, and methods for transferring, Storing, and/or dispensing a fluid, such as an ocular drug. Some embodiments advantageously Provide devices, systems, and methods for reconstituting lyophilized materials and Administering the reconstituted lyophilized product to a delivery location. In one embodiment, a device includes: a first end; a second end opposite the first end; and a chamber extending between the first end and the second end, the first end being configured to be engageable with a secondary reservoir to accept a fluid from the secondary reservoir and into the chamber, and the second end being configured to release a fluid from the chamber to a delivery location.

In some aspects, the device further includes a body, the body including the first end and the second end and at least partially defining the chamber.

In some aspects, the first end includes an engagement assembly configured to be engageable with the secondary reservoir.

In some aspects, the first end includes a recessed portion that is sized and configured to contain the engagement assembly therein.

In some aspects, the engagement assembly includes: a stopper; and a retaining cap, the retaining cap retaining the stopper within the recessed portion when the device is assembled.

In some aspects, the stopper is composed of a self-sealing material.

In some aspects, the retaining cap includes an aperture, the aperture exposing at least a portion of the stopper.

In some aspects, the first end includes a flange, the flange at least partially defining the recessed portion.

In some aspects, the device further includes at least one delivery aperture at the second end.

In some aspects, the second end at least partially defines the at least one delivery aperture.

In some aspects, the device further includes a cap, at least a portion of the cap being engaged with the second end when the device is assembled.

In some aspects, the body is composed of a resiliently deformable material.

In some aspects, a device for reconstituting a lyophilized material includes: a body including a first end, a second end opposite the first end, and a chamber extending between the first end and the second end, the first end having a first opening and the second end having a second opening, each of the first opening and the second opening being in fluid communication with the chamber; an engagement assembly coupled to the first end and being in fluid communication with the first opening; and at least one delivery aperture at the second end and being in fluid communication with the second opening.

In some aspects, the device further includes a cap, at least a portion of the cap being engageable with the second end.

In some aspects, the second opening of the second end of the body defines the at least one delivery aperture.

In some aspects, the first end defines a recessed portion, the engagement assembly being entirely within the recessed portion when the device is assembled, the engagement assembly including: a stopper; and a retaining cap, the stopper being between the first opening and the retaining cap when the device is assembled, the retaining cap defining an aperture that exposes at least a portion of the stopper therethrough.

In some aspects, a method of reconstituting a powdered material includes: engaging a secondary reservoir with an engagement assembly of a delivery device, the delivery device defining a first end having the engagement assembly and a first opening, a second end having at least one delivery aperture, and a chamber extending between the first end and the second end, the chamber containing the powdered material; and delivering a fluid from the secondary reservoir through the engagement assembly and into the chamber to reconstitute the powdered material.

In some aspects, the method further includes: delivering the reconstituted powdered material to a delivery location through the at least one delivery aperture.

In some aspects, the engagement assembly includes: a stopper; and a retaining cap, the stopper being between the first opening and the retaining cap when the delivery device is assembled, the retaining cap defining an aperture that exposes at least a portion of the stopper therethrough.

In some aspects, the secondary reservoir is a hypodermic syringe having a needle, the step of engaging the secondary reservoir with the engagement assembly including inserting at least a portion of the needle through the aperture of the retaining cap, through the stopper, and into the chamber of the delivery device.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a top perspective view of an exemplary embodiment of a fluid transfer and dispensing device, in accordance with the present disclosure;

FIG. 2 shows a bottom perspective of the device of FIG. 1, in accordance with the present disclosure;

FIGS. 3 and 4 show opposite side views of the device of FIG. 1, in accordance with the present disclosure;

FIG. 5 shows a cross-sectional view of the device of FIG. 1, in accordance with the present disclosure;

FIG. 6 shows an exploded view of the device of FIG. 1 in accordance with the present disclosure;

FIG. 7 shows an exemplary embodiment of a fluid transfer and dispensing device in use with a secondary reservoir, in accordance with the present disclosure;

FIG. 8 shows an exemplary method for using a fluid transfer and dispensing device, in accordance with the present disclosure; and

FIGS. 9A and 9B show a further exemplary method of using a fluid transfer and dispensing device, in accordance with the present disclosure.

### DETAILED DESCRIPTION

Before describing in detail exemplary embodiments, it is noted that the embodiments reside primarily in combinations of apparatus components and steps related to the administration of a product to a delivery location and, in some embodiments, the reconstitution and administration of lyophilized products. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

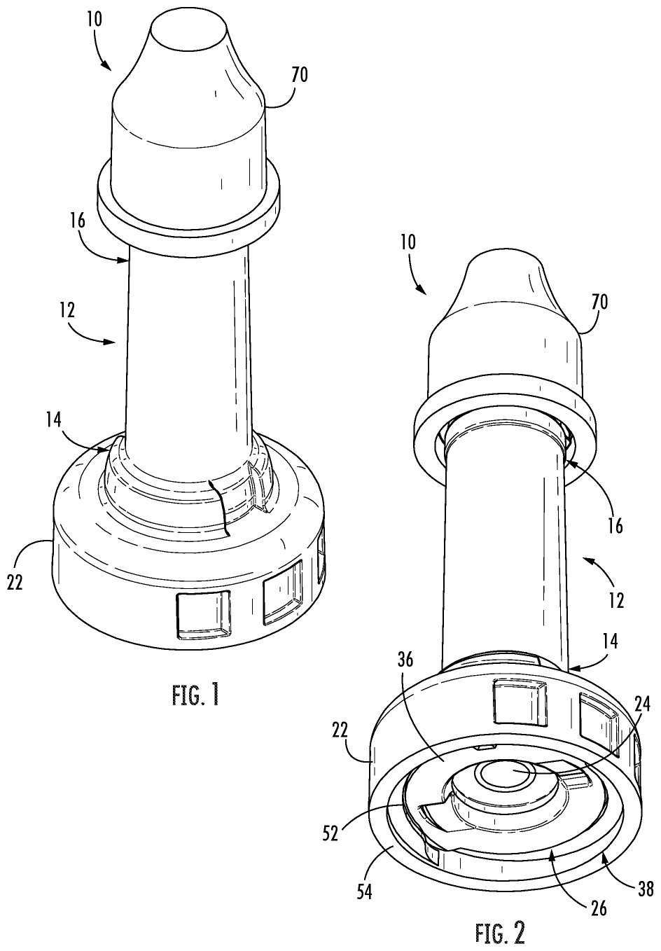

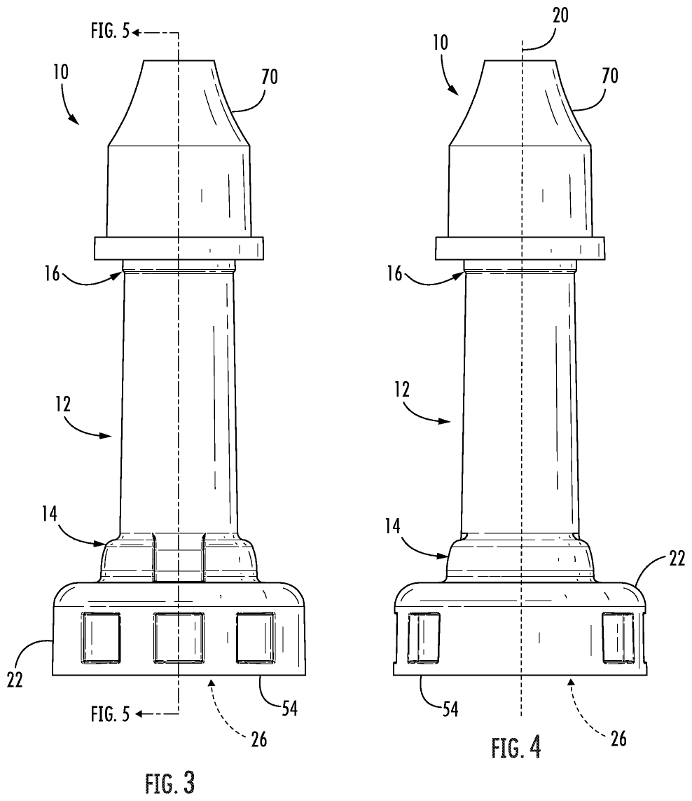

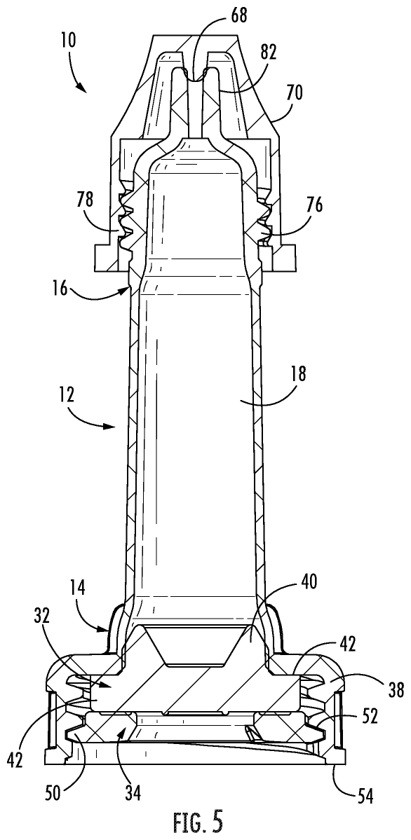

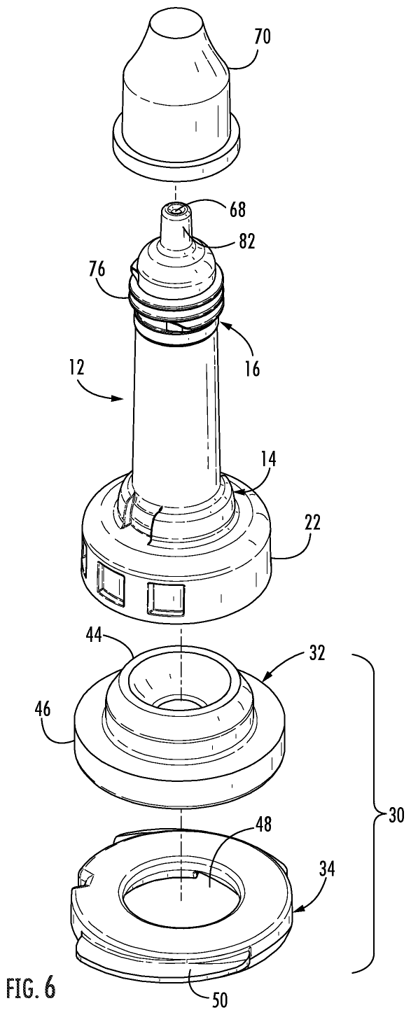

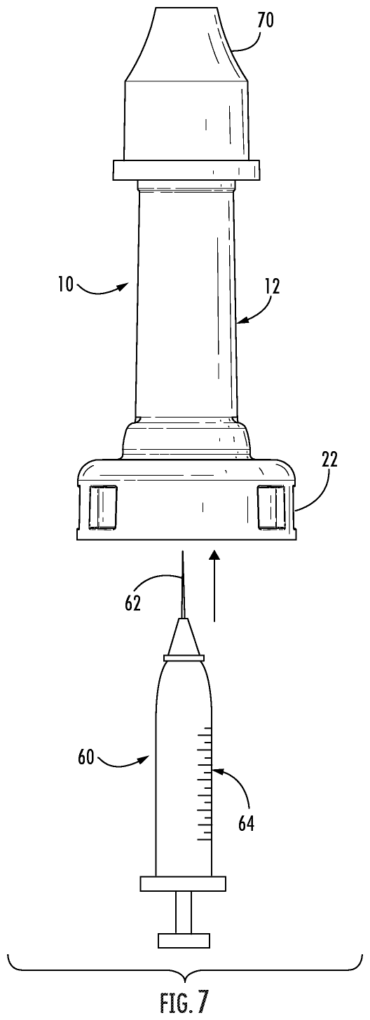

Referring now to the figures in which like reference designators are used for like elements, an exemplary embodiment of a fluid transfer and dispensing device 10 (referred to herein as a “device”) is shown in FIGS. 1-7. FIG. 1 shows a top perspective view of the device, FIG. 2 shows a bottom perspective view of the device, FIGS. 3 and 4 show side views of the device, FIG. 5 shows a cross-sectional view of the device, FIG. 6 shows an exploded view of the device, and FIG. 7 shows an exemplary embodiment of a fluid transfer and dispensing device in use with a secondary reservoir. In some embodiments, the device is configured for reconstituting a lyophilized material and administering a reconstituted lyophilized product to a delivery location. However, it will be understood that the device may be configured to accept any fluid (such as a liquid, gas, gel, or the like) at a first end of the device and to administer the fluid, without or without being mixed with another product within the device, such as a lyophilized product, from a second end of the device. Further, although the term “lyophilized” refers to a material that has been freeze dried, and the devices and methods disclosed herein are suitable for use with lyophilized materials, it will be understood that the devices and methods disclosed herein may additionally or alternatively may be used for materials, such as other powdered materials, that are not lyophilized but that require reconstitution and/or mixing with a fluid.

Continuing to refer to FIGS. 1-6, the device 10 generally includes a body 12 having a first end 14 and a second end 16 opposite the first end 14. In general, fluid may be introduced into the device 10 through the first end 14 and the fluid may be delivered or administered from the device 10 through a second end 16. In one non-limiting example, the fluid is mixed with at least one other ingredient within the device 10, such as a lyophilized material, and a reconstituted lyophilized product may be delivered or administered from the device 10 through the second end 16. Regardless of whether the fluid is mixed with one or more other ingredients, it will be understood that the device 10 is configured to both accept the fluid and to administer the fluid while preserving sterility of the fluid and preventing the introduction of contaminants into the device and/or fluid.

Continuing to refer to FIGS. 1-6, in one embodiment the body 12 is elongate and defines a chamber 18 therein, and the body 12 defines a longitudinal axis 20. As shown in the figures, at least a portion of the body 12 may have a tubular or at least substantially tubular shape between the first end 14 and the second end 16. In one non-limiting example, the device 10 is sold, transported, and/or stored with a treatment product within the chamber 18, such as lyophilized or powdered material, or a fluid, such as one or more liquids, gases, gels, or the like. In another non-limiting example. the device 10 is sold, transported, and/or stored with an empty chamber 18.

Continuing to refer to FIGS. 1-6, in one embodiment the first end 14 of the body 12 includes a flange 22. The flange 22 may be coupled to, affixed to, extend from, and/or integrated with the first end 14 of the body 12. In one embodiment, the flange 22 is configured to allow the device 10 to stably stand upright when the flange 22 is placed on a surface, with the body 12 and second end 16 extending away and orthogonal to, or at least substantially orthogonal, to the surface. For example, in one embodiment the flange 22 has an outer diameter that is greater than an outer diameter of the body 12 (that is, the flange 22 is wider than at least one other portion of the body 12), which gives stability to the device 10 when placed to stand upright on a surface.

Continuing to refer to FIGS. 1-6, in one embodiment the first end 14 of the body 12 defines a first opening 24, and is an open end when the device 10 is disassembled. However, it will be understood that other configurations may be used. In one embodiment, the first end 14 further includes a recessed portion 26 that is at least partially defined by the interior of the flange 22, and the first opening 24 of the first end 14 is located within or in fluid communication with the recessed portion 26 of the flange 22. The recessed portion 26 is sized and configured to securely retain therein an engagement assembly 30. In one embodiment, the engagement assembly 30 includes a stopper 32 and a retaining cap 34. In one embodiment, the recessed portion 26 is at least partially defined by an inner engagement surface 36 that extends around the first opening 24 and a circumferential wall 38 that extends around the circumference of the inner engagement surface 36 and extends orthogonally to, or at least substantially orthogonal to, the inner engagement surface 36. In one non-limiting example, the circumferential wall 38 at least partially defines the flange 22 and extends away from the first end 14 of the body 12 in a direction that is parallel to, or at least substantially parallel to, the longitudinal axis 20 of the body 12. In some embodiments, the inner engagement surface 36 includes a neck portion 40 and a shoulder portion 42. In some embodiments, the shoulder portion 42 extends in a direction that is orthogonal to, or at least substantially orthogonal to, the longitudinal axis 20 of the body 12 to meet the circumferential wall 38.

Continuing to refer to FIGS. 1-6, in one embodiment. the stopper 32 is composed of a self-sealing material such as natural or synthetic rubber, silicone, resilient polymer, or other material that is configured to provide a fluid-and air-tight seal, even after puncture or perforation by the needle of a syringe (for example, a hypodermic syringe) or other instrument. In some embodiments, the stopper 32 is sized and configured such that a first portion 44 of the stopper 32 is insertable into the first end 14 of the body 12 with the first portion 44 within the neck portion 40 of the inner engagement surface 36 and engaged therein by friction fit, such that the first portion 44 abuts or is located proximate the first opening 24, and a second portion 46 of the stopper 32 abuts or is in contact with the shoulder portion 42 of the inner engagement surface 36 to effectively seal the chamber 18 of the body 12 and prevent materials within the chamber 18 from escaping from the device 10.

Continuing to refer to FIGS. 1-6, and with reference to FIG. 7, in one embodiment, the retaining cap 34 is a generally planar and generally disk-shaped, and includes an aperture 48 and at least one tab 50 on the outer edge of the disk. In one embodiment, the circumferential wall 38 of the recessed portion 26 of the flange 22 includes threading or other structural feature 52 that is sized and configured to receive therein at least a portion of each tab 50 on the retaining cap 34. In some embodiments, when assembled, the stopper 32 is seated within the first end 14 of the body 12 and the retaining cap 34 is secured within the recessed portion 26 of the flange 22 (for example, by fixed or removable engagement between the tabs 50 of the retaining cap 34 and the circumferential wall 38 of the recessed portion 26) to securely retain the stopper 32 to the body 12. However, it will be understood that other structural features may be used to secure the retaining cap 34 within the recessed portion 26, such as clamps, clips, threading, ridges, tabs and notches, followers and grooves, or the like. Further, at least a portion of the stopper 32 is exposed or accessible through the aperture 48 in the retaining cap 34. When assembled, the retaining cap 34 does not extend beyond the recessed portion 26. In one embodiment, the retaining cap 34 and/or the planar rim 54 of the flange 22 define a planar surface that allows the device 10 to stably stand upright on a surface. As is discussed in greater detail below, fluid may be added to the chamber 18 of the body 12 by engaging a secondary reservoir 60 to the body 12, such as by inserting the needle 62 of a syringe 64 through the aperture 48 of the retaining cap 34 and through the stopper (for example, as shown in FIG. 7). Fluid or other materials may then be delivered from the secondary reservoir 60 into the chamber 18. In one embodiment, when a syringe with needle 62 is used and the needle 62 is removed after the delivery of fluid into the chamber 18, the puncture in the stopper 32 self-seals to again create a fluid-tight seal. Further, the retaining cap 34 prevents movement or dislodgement of the stopper 32 when the needle 62 is withdrawn from the stopper 32.

Continuing to refer to FIGS. 1-6, in one embodiment the second end 16 of the body 12 defines a second opening 68. In one embodiment the device 10 further includes a cap 70, at least a portion of which being removably engageable with the second end 16 of the body 12. The cap 70 is sized and configured to receive therein at least a portion of the second end 16 of the body 12, including the second opening 68. In some embodiments, the cap 70 is removably engaged or engageable with the second end 16 of the body 12. In other embodiments, the entire cap 70 is permanently engaged or engageable with the second end 16 of the body 12. Additionally, in some embodiments, the second end 16 of the body 12 includes threading and/or other structural features 76 (for example, clamps, ridges, tabs and notches, followers and grooves, or the like) for securely and permanently and/or removably engaging at least a portion of the cap 70. In some embodiments, the cap 70 includes one or more structural features 78 (for example, threading clamps, ridges, tabs and notches, followers and grooves, or the like) for securely and permanently or removably engaging one portion of the cap 70 with another portion of the cap 70. In one non-limiting example, the cap may be screwed onto the second end 16 of the body (by engaging threading 76 of the body 12 and threading 78 of the cap 70) to conceal and protect the second opening 68 and to prevent materials within the chamber 18 from escaping from the device 10.

Continuing to refer to FIGS. 1-6, in some embodiments, at least a portion of the second end 16 of the body 12 is configured for administering a product including, but not limited to, a lyophilized product such as a reconstituted lyophilized product. In one embodiment, the second opening 68 of the second end 16 defines a delivery aperture that is configured as a dropper tip, a spray tip, a free-flow aperture, a dauber, or have another configuration or feature that allows a user to deliver the product from the second end 16 of the body 12 of the device 10 to a delivery location. In one non-limiting example, the second end defines a neck portion 82 that includes the second opening 68 as the delivery aperture, similar to an eye dropper (for example, as shown in FIGS. 5 and 6). In some embodiments, at least a portion of the cap 70 is configured to be permanently or removably attached to the second end 16 of the body 12 and that defines the delivery aperture 68, whereas another portion of the cap 70 is configured to be removably attached to the second end 16 of the body 12 and/or the other portion of the cap 70 and to conceal the delivery aperture 68. In some embodiments, the second opening 68 is structurally separate from a delivery aperture, but is in fluid communication with and works in concert with the delivery aperture. It will be understood that a cap 70 and/or body 12 having any configuration suitable for the product to be administered, method of administration, or other consideration, and that is still capable of preventing contamination of materials within the chamber 18, may be used. Additionally, the cap 70 and/or body 12 may include one or more structural features proximate the at least one delivery aperture that enable the device to be coupled to a delivery component, such as a needle for injecting the product at a delivery location. The cap 70 may be sealed before use, such as with a tamper-proof or tamper-evident seal, hermetic seal, protected with a shrink wrap or band, or the like, which prevents contamination of materials within the chamber 18 of the body 12 and/or indicates if the cap 70 has been removed from the body 12.

Continuing to refer to FIGS. 1-6, in one embodiment the body 12 is composed of a semi-rigid and/or resiliently deformable material that allows a user to squeeze the body 12 to release, administer, eject, dispense, or otherwise deliver fluid contents from within the chamber 18 through the at least one delivery aperture 68 to a delivery location. For example, the body 12 may be composed of low-density polyethylene (LDPE) or similar material, and may be opaque, translucent, or transparent. In other embodiments, the body 12 may be composed of a more rigid material, such as high-density polyethylene (HDPE), polypropylene (PP), or similar material, such as when the device 10 is used to spray fluid contents or to release fluid contents without the need to squeeze the body 12. Thus, in one non-limiting example, the device 10 may be used as an eye dropper to deliver fluid drops from the chamber 18 through the at least one delivery aperture 68 at the second end 16 of the body 12 to a patient's eye. It will also be understood that the body 12, and other components of the device 10, may be composed of the same or different materials, or combinations of materials.

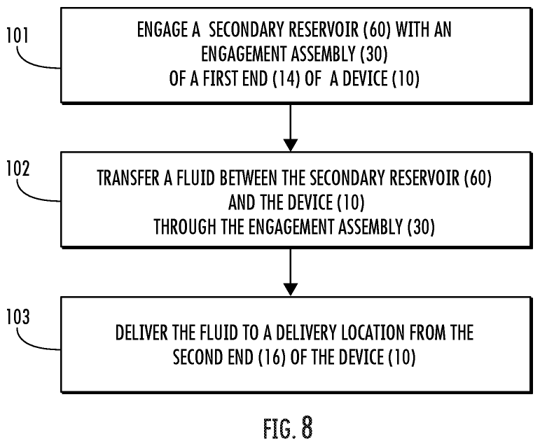

Referring now to FIG. 8, an exemplary method of using a fluid transfer and dispensing device is shown. In one embodiment, the device 10 may be used to receive one or more ingredients of a product and to store the product before use, as well as to administer the product, in a sterile manner.

Continuing to refer to FIG. 8, and with reference to FIG. 7, in an exemplary first step 101, a secondary reservoir 60 is engaged with the first end 14 of the body 12 of the device 10. The chamber 18 may or may not already contain a fluid, gel, semi-solid, solid, gas, or other material therein. In some embodiments, such as when the device 10 is as shown and described in FIGS. 1-6 and the secondary reservoir 60 is a syringe 64 having a needle 62, the user inserts the needle 62 of the syringe 64 through the aperture of the retaining cap 34, through the stopper 32, and into the chamber 18 of the body 12 (for example, as shown in FIG. 7). The secondary reservoir 60 may be any device configured to retain a fluid or other material, at least temporarily, and pass it into the device 10 described herein, and is not limited to the exemplary secondary reservoir 60 shown in FIG. 7. Additionally or alternatively, the secondary reservoir may be device configured to retain a fluid, a gel, a semi-solid, solid, and/or a gas, at least temporarily, and pass it into the device described herein. For example, the secondary reservoir 60 may be configured to retain a powdered or lyophilized material therein and to pass the material into the device 10 when coupled to the engagement assembly 30.

Continuing to refer to FIG. 8, in an exemplary second step 102, which may be performed by the same or different user than the first step 101, fluid or other material is injected, passed, or otherwise delivered from the secondary reservoir 60 into the chamber 18 of the body 12 of the device 10. In some embodiments, a fluid or other material is already contained within the chamber 18, where it mixes with the fluid or other material passed from the secondary reservoir 60. In other embodiments, the chamber 18 of the device is empty before being coupled to the secondary reservoir 60, and the device 10 is used merely to transfer materials from the secondary reservoir 60 to the delivery location in a sterile manner. In embodiments wherein the device 10 is as shown and described in FIGS. 1-7 and the secondary reservoir 60 is a syringe with or without a needle 62, the user depresses the plunger of the syringe 64 to inject fluid from the syringe body 64 into the chamber 18. Optionally, the user may roll, rotate, and/or shake the device 10, or otherwise agitate the material(s) within the chamber 18, to help ensure sufficient mixing of materials. Once all of the one or more materials are within the chamber 18, the material or mixture of materials to be administered is referred to herein as the “product.”

Continuing to refer to FIG. 8, in an optional exemplary third step 103, which may be performed by the same or different user than the first and second steps 101, 102, the product is administered or delivered to the delivery location. In some embodiments, the product is delivered through at least one delivery aperture (for example, the second opening 68) within or integrated with the second end 16 of the body 12 (or another delivery aperture within or integrated with the second end 16 and/or the cap 70). In one non-limiting example, the device 10 may include a single delivery aperture 68 that is configured as a dropper. In such an example, a user may squeeze the body 12 of the device 10 to release a drop of the product from the second end 16 of the body 12 of the device 10. In another non-limiting example, the device 10 may include a single delivery aperture 68 that is configured as a spray nozzle. In such an example, a user may depress a portion of the spray nozzle to eject a spray of droplets of product from the second end 16 of the body 12 of the device 10.

Continuing to refer to FIG. 8, the method shown is a generalized method of use of the device 10, which may encompass any of a variety of embodiments of use. As a first non-limiting example, the secondary reservoir 60 is a syringe 64 containing a liquid and the engagement assembly 30 of the device includes a stopper 32 (for example, as shown in FIGS. 5 and 6). The chamber 18 of the device 10 may contain a material, such as a powdered or lyophilized material, or may be empty prior to coupling the secondary reservoir 60 to the device 10. The liquid from the secondary reservoir 60 may be suitable for reconstituting a powdered or lyophilized material within the chamber 18, or may be another fluid, such as a liquid product for administration without being mixed with another ingredient or material. Once the powdered or lyophilized material is adequately mixed (in embodiments wherein the chamber contains a powdered or lyophilized material), the reconstituted product is administered from the second end 16 of the device 10 to the delivery location. The liquid may be saline, water, or other biologically compatible liquid, flowable gel, or similar fluids, and/or combinations thereof, and may optionally include one or more buffers, carriers, additives, stabilizers, or other ingredients. Additionally or alternatively, the liquid may be ready for administration as is, without mixing with another material or ingredient. As a second non-limiting example, the secondary reservoir 60 is a vial containing a powdered or lyophilized material and the chamber of the device contains a liquid, such as a diluent. The engagement assembly 30 may include a double-ended transfer spike or a single-ended transfer spike with luer lock connection. After the secondary reservoir 60 is coupled to the device 10, liquid from the chamber 18 is passed into the secondary reservoir 60 (such as by gravity), where the liquid reconstitutes the powdered or lyophilized material. Once adequately mixed, the reconstituted product is transferred back to the chamber 18 (such as by gravity), from where the product may be administered from the second end 16 of the device 10 to the delivery location. As a third non-limiting example, the secondary reservoir 60 is a vial or syringe containing a liquid, such as a diluent, and the chamber contains a powdered or lyophilized material. The engagement assembly 30 may include a stopper 32, a double-ended transfer spike, or a single-ended transfer spike with luer lock connection. After the secondary reservoir 60 is coupled to the device 10, liquid from the secondary reservoir 60 is passed into the chamber 18 of the device 10, where the liquid reconstitutes the powdered or lyophilized material. Once adequately mixed, the reconstituted product is administered from the second end 16 of the device 10 to the delivery location. As a fourth non-limiting example, the secondary reservoir 60 is a vial or syringe containing a first liquid and the chamber contains a second liquid. The engagement assembly 30 may include a stopper, a double-ended transfer spike, or a single-ended transfer spike with luer lock connection. After the secondary reservoir 60 is coupled to the device 10, the first liquid from the secondary reservoir 60 is passed into the chamber 18 of the device 10, where the first and second liquids mix. Once adequately mixed, the product is administered from the second end 16 of the device 10 to the delivery location. In addition to these non-limiting examples, it will be understood that the devices and methods described herein may have other suitable configurations, components, and/or steps for the sterile transfer and delivery of a product to a delivery location.

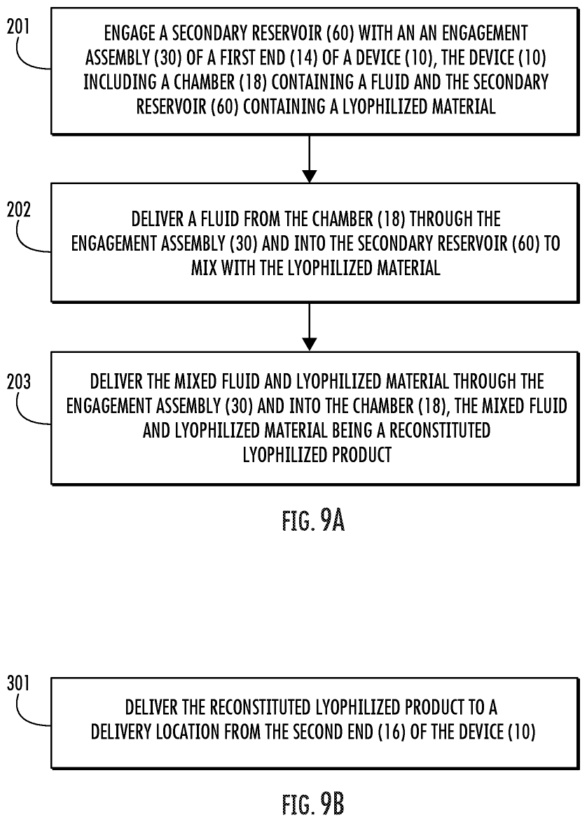

Referring now to FIGS. 9A and 9B, an exemplary method of using a fluid transfer and dispensing device is shown. FIG. 9A shows a method of reconstituting a lyophilized material and FIG. 9B shows a method of administering a reconstituted lyophilized product to a delivery location. The method steps shown in FIGS. 9A and 9B are non-limiting examples of the general method steps shown in FIG. 8. Further, the method steps shown in FIGS. 9A and 9B may be performed by the same person or different people, and at a same time and/or location or different times and/or locations. Before administration, the device 10 may be used to store the lyophilized material or reconstituted lyophilized product in a sterile manner. The steps shown in FIGS. 9A and 9B may be performed by the same or different user. For example, the lyophilized material may be reconstituted immediately before administration, and the reconstitution and administration may be performed by the same user. In another example, the lyophilized material may be reconstituted by a first user and the reconstituted lyophilized material may be administered by a second user. In one non-limiting example, the device 10 may be used to administer a reconstituted lyophilized product to an eye, but it will be understood that the device is not so limited in its use and/or product may be delivered to other delivery locations. As noted above, in some embodiments the device 10 is sold, transported, and/or stored already containing the lyophilized material within the chamber 18. However, it will be understood that the device 10 may instead be sold, transported, and/or stored empty and the lyophilized material added before use. Additionally, it will be understood that the device 10 may be used to mix a fluid with a material other than a lyophilized or powdered material, such as one or more additional fluids and/or other ingredients, within the device in a sterile manner. For illustration, however, use of the device for reconstituting a lyophilized material is shown in FIGS. 9A and 9B.

Referring to FIG. 9A, in an exemplary first step 201 of reconstituting a lyophilized material, a secondary reservoir 60 is engaged with the first end 14 of the body 12 of the device 10 (for example, as discussed above regarding FIG. 8). In one embodiment, the secondary reservoir 60 contains a powdered or lyophilized material and the chamber 18 of the device 10 contains a liquid, such as a diluent. In some embodiments, such as when the device 10 is as shown and described in FIGS. 1-7 and the secondary reservoir 60 is a syringe 64 having a needle 62, the user inserts the needle 62 of the syringe 64 through the aperture 48 of the retaining cap 34, through the stopper 32, and into the chamber 18 of the body 12. In other embodiments, the secondary reservoir 60 is a vial and the engagement assembly 30 includes a double-ended transfer spike or single-ended transfer spike and luer lock connection. The secondary reservoir 60 may be any device configured to retain a fluid or other material, at least temporarily, and pass it into the device 10 described herein.

Continuing to refer to FIG. 9A, in an exemplary second step 202 of reconstituting a lyophilized material, which may be performed by the same or different user than the first step 201, a liquid is passed or delivered, such as by gravity, from the chamber 18 of the body 12 into the secondary reservoir 60. Optionally, the user may roll, rotate. and/or shake the coupled device 10 and secondary reservoir 60, or otherwise agitate the material(s) within the secondary reservoir 60, to help ensure sufficient mixing of materials and reconstitution of the lyophilized material.

Continuing to refer to FIG. 9A, in an exemplary third step 203 of reconstituting lyophilized material, which may be performed by the same or different user than the first and second steps 201, 202, the user inverts the coupled device 10 and allows the resulting mixture to pass from the secondary reservoir 60 and into the chamber 18 of the device 10, such as by gravity, injection, or other means, depending on the configuration of the secondary reservoir 60. Optionally, the user may roll, rotate, and/or shake the coupled device 10 and secondary reservoir 60, or otherwise agitate the material(s) within the chamber 18. Once the lyophilized material and the liquid are within the chamber 18 and the lyophilized material is reconstituted, the mixture to be administered is referred to herein as the reconstituted lyophilized product.

Referring to FIG. 9B, an exemplary step 301 of administering a reconstituted lyophilized product to a delivery location is shown. In some embodiments, the product is delivered through at least one delivery aperture 68 within or integrated with second end 16 of the body 12 (and/or the cap 70). In one non-limiting example, the device 10 may include a single delivery aperture 68 that is configured as a dropper. In such an example, a user may squeeze the body 12 of the device 10 to release a drop of the reconstituted lyophilized product from the second end 16 of the body 12 of the device 10. In another non-limiting example, the device 10 may include a single delivery aperture 68 that is configured as a spray nozzle. In such an example, a user may depress a portion of the spray nozzle to eject a spray of droplets of reconstituted lyophilized product from the second end 16 of the body 12 of the device 10. However, it will be understood that the second end 16 of the device 10 may include one or more delivery apertures configured for a particular method of administration, in addition to or instead of a dropper or spray nozzle, such as a free-flow aperture, a dauber, needle connection, or other configuration or feature that allows a user to deliver the product from the second end 16 of the body 12 of the device 10 to a delivery location.

Thus, the device disclosed herein is generally configured to receive a fluid through a first end of the device and to deliver or administer the fluid through a second end of the device. Once received within the device, the sterility of the fluid is preserved because the first and second ends remain closed until use. Additionally, the fluid may mix with one or more additional ingredients within the device prior to administration, such as one or more liquids, gases, gels, or solids. In one non-limiting example, the device is configured to contain a lyophilized material within a chamber within the device. The device is additionally configured to be engaged with a secondary reservoir and to accept fluid or other material from the secondary reservoir through a first end of the device to reconstitute the lyophilized material, the first end being in fluid communication with the chamber. The device is additionally configured to administer, deliver, or release the reconstituted lyophilized material from a second end of the device to a delivery location, the first end being opposite the second end and in fluid communication with the chamber. The device disclosed herein may be used to store the lyophilized material for extended periods of time, and then may be used to reconstitute the lyophilized material before administration to the delivery location (for example, immediately before administration).

It will also be understood that the device may be used for method(s) other than those shown and described herein. In one non-limiting example, the chamber may contain a first fluid (for example, a diluent) and the secondary reservoir may contain a solid (for example, a powdered material) or a second fluid (for example, a liquid or gel). The first end of the device may then be used to deliver the first fluid into the secondary reservoir to mix with and reconstitute the solid and/or mix with the second fluid, and the resulting mixture may then be transferred from the secondary reservoir back into the chamber of the device, either by simply inverting the coupled device and secondary reservoir (to pass the resulting mixture through the second end of the device) or by the method as shown and described in FIG. 5 (to pass the resulting mixture through the first end of the device). Finally, the device may be used to administer the resulting mixture to a delivery location. Other methods of use that preserve sterility of the product to be administered are also contemplated.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

### EMBODIMENTS

In one embodiment, a device includes: a first end; and a second end opposite the first end, the first end being configured to be engageable with a secondary reservoir and the second end being configured to release a fluid therefrom.

In one aspect of the embodiment, the device further includes: a body, the body extending between the first end and the second end; and a chamber, the chamber defined within the body.

In one aspect of the embodiment, the first end includes an engagement assembly configured to be engageable with the secondary reservoir.

In one aspect of the embodiment, the first end includes a recessed portion that is sized and configured to contain the engagement assembly therein.

In one aspect of the embodiment, the engagement assembly includes: a stopper; and a retaining cap, the retaining cap retaining the stopper within the recessed portion when the device is assembled.

In one aspect of the embodiment, the stopper is composed of a self-sealing material.

In one aspect of the embodiment, the retaining cap includes an aperture, the aperture exposing at least a portion of the stopper.

In one aspect of the embodiment, the engagement assembly includes a one-way valve, the one-way valve being in fluid communication with the chamber.

In one aspect of the embodiment, the first end includes a flange, the flange at least partially defining the recessed portion.

In one aspect of the embodiment, the device further includes at least one delivery aperture at the second end.

In one aspect of the embodiment, the second end at least partially defines the at least one delivery aperture.

In one aspect of the embodiment, the device further includes a cap, at least a portion of the cap being engaged with the second end when the device is assembled.

In one aspect of the embodiment, at least a portion of the cap defines the at least one delivery aperture.

In one aspect of the embodiment, the body is composed of a resiliently deformable material.

In one aspect of the embodiment, the device further includes a venting valve at at least one of the first end and the second end.

In one embodiment, a device for reconstituting a lyophilized material includes: a body including a first end, a second end opposite the first end, and a chamber extending between the first end and the second end, the first end having a first opening and the second end having a second opening. each of the first opening and the second opening being in fluid communication with the chamber; an engagement assembly coupled to the first end and being in fluid communication with the first opening; and at least one delivery aperture at the second end and being in fluid communication with the second opening.

In one aspect of the embodiment, the device further includes a cap, at least a portion of the cap being engageable with the second end.

In one aspect of the embodiment, at least a portion of the cap defines the at least one delivery aperture.

In one aspect of the embodiment, the first end defines a recessed portion, the engagement assembly being entirely within the recessed portion, the engagement assembly including: a stopper; and a retaining cap, the stopper being between the first opening and the retaining cap, the retaining cap defining an aperture that exposes at least a portion of the stopper therethrough.

In one embodiment, a method of reconstituting a powdered material includes: engaging a secondary reservoir with an engagement assembly, the device defining a first end having the engagement assembly, a second end having at least one delivery aperture, and a chamber, the first end being opposite the second end, the chamber containing the powdered material; and delivering a fluid from the secondary reservoir through the engagement assembly and into the chamber to reconstitute the powdered material.

In one aspect of the embodiment, the method further includes delivering the reconstituted powdered material to a delivery location through the at least one delivery aperture.

1. A device, comprising:
a first end;
a second end opposite the first end; and
a chamber extending between the first end and the second end, the first end being configured to be engageable with a reservoir to accept a fluid from the reservoir and into the chamber, and the second end being configured to release a fluid from the chamber to a delivery location.

2. The device of claim 1, wherein the device further comprises a body, the body including the first end and the second end and at least partially defining the chamber.

3. The device of claim 1, wherein the first end includes an engagement assembly configured to be engageable with the reservoir.

4. The device of claim 3, wherein the first end includes a recessed portion that is sized and configured to contain the engagement assembly therein.

5. The device of claim 4, wherein the engagement assembly includes:
a stopper; and
a retaining cap, the retaining cap retaining the stopper within the recessed portion when the device is assembled.

6. The device of claim 5, wherein the stopper is composed of a self-sealing material.

7. The device of claim 5, wherein the retaining cap includes an aperture, the aperture exposing at least a portion of the stopper.

8. The device of claim 4, wherein the first end includes a flange, the flange at least partially defining the recessed portion.

9. The device of claim 1, wherein the device further comprises at least one delivery aperture at the second end.

10. The device of claim 9, wherein the second end at least partially defines the at least one delivery aperture.

11. The device of claim 9, wherein the device further comprises a cap, at least a portion of the cap being engaged with the second end when the device is assembled.

12. The device of claim 2, wherein the body is composed of a resiliently deformable material.

13. A device for reconstituting a lyophilized material, the device comprising:
a body including a first end, a second end opposite the first end, and a chamber extending between the first end and the second end, the first end having a first opening and the second end having a second opening, each of the first opening and the second opening being in fluid communication with the chamber;
an engagement assembly coupled to the first end and being in fluid communication with the first opening; and
at least one delivery aperture at the second end and being in fluid communication with the second opening.

14. The device of claim 13, further comprising a cap, at least a portion of the cap being engageable with the second end.

15. The device of claim 14, wherein the second opening of the second end of the body defines the at least one delivery aperture.

16. The device of claim 14, wherein the first end defines a recessed portion, the engagement assembly being entirely within the recessed portion when the device is assembled, the engagement assembly including:
a stopper; and
a retaining cap, the stopper being between the first opening and the retaining cap when the device is assembled, the retaining cap defining an aperture that exposes at least a portion of the stopper therethrough.

17. A method of reconstituting a powdered material, the method comprising:
engaging a reservoir with an engagement assembly of a delivery device, the delivery device defining a first end having the engagement assembly and a first opening, a second end having at least one delivery aperture, and a chamber extending between the first end and the second end, the chamber containing the powdered material; and
delivering a fluid from the reservoir through the engagement assembly and into the chamber to reconstitute the powdered material.

18. The method of claim 17, the method further comprising:
delivering the reconstituted powdered material to a delivery location through the at least one delivery aperture.

19. The method of claim 17, wherein the engagement assembly includes:
a stopper; and
a retaining cap, the stopper being between the first opening and the retaining cap when the delivery device is assembled, the retaining cap defining an aperture that exposes at least a portion of the stopper therethrough.

20. The method of claim 19, wherein the reservoir is a hypodermic syringe having a needle, the step of engaging the reservoir with the engagement assembly including inserting at least a portion of the needle through the aperture of the retaining cap, through the stopper, and into the chamber of the delivery device.
